(19) United States
(12) Patent Application Publication
(10) Pub. No.: US 2013/0102913 A1
(43) Pub. Date: Apr. 25, 2013

(54) Patient Readable Portable Atrial Fibrillation Detector

(71) Applicant: Vernon N. Reaser, Victoria, TX (US)

(72) Inventor: Vernon N. Reaser, Victoria, TX (US)

(21) Appl. No.: 13/385,594

(22) Filed: Oct. 18, 2011

(51) Int. Cl. A61B 5/0245; A61B 7/00; A61B 5/00; A61B 5/0456; A61B 5/0472; A61B 5/04; A61B 5/046; A61B 5/025

(52) U.S. Cl. CPC A61B 5/0245; A61B 5/046; A61B 7/00; A61B 5/025; A61B 5/0456; A61B 5/0472; A61B 5/04012; A61B 5/742; A61B 5/6823; A61B 5/0006; A61B 5/6824
USPC 600/518; 600/528; 600/527

(57) ABSTRACT

Systems and devices to gather data from a subject's heart, analyze said data to determine whether the subject is experiencing cardiac arrhythmia, and display results of said determining. Use, and display of cardiac condition information, are preferably simple and unambiguous to untrained users.

### BACKGROUND

Atrial fibrillation is the most common form of cardiac arrhythmia, and involves the two upper chambers of the heart. A trained medical technician or Doctor can usually recognize the unique heart contractions related to Atrial Fibrillation or Atrial Flutter. Trained medical personnel can generally detect Atrial Fibrillation by taking a patient's pulse, but it takes training and experience that most lay persons lack. Typically, a doctor will use a 12-lead EKG to make a definitive determination of Atrial Fibrillation or Atrial Flutter.

No device to date provides individuals with unaided rapid determination of whether they are experiencing Atrial Fibrillation or Atrial Flutter, which left untreated for longer than 48 hours can lead to risk of debilitating stroke or death.

The costs of health care are rising quickly, and rapid access to emergency health services is perennially uncertain.

For ease of reading Artial Fibulation and Atrial Flutter will be collectively referred as Afib.

Generally, individuals are at high risk to develop Afib beginning at age 65. Ten thousand people per day turn 65 in the U.S. alone. Other people at risk of developing Afib include adolescents, due to the increasing popularity of energy drinks; diabetics, who have a 40% higher risk of developing Afib than those without diabetes; and those who have a close family member with Afib have a 40% higher risk of developing Afib.

Of people with untreated Afib, 7%, if left untreated, will die or be permanently and seriously disabled, likely requiring full time medical care.

Afib can be treated successfully in almost all cases, if the victim is properly and timely screened. Screening is far less expensive than treatment of debilitating strokes and other symptoms of Afib that is not timely treated.

Millions of people—potentially 30 million in the U.S.—are already at risk, and therefore in need of screening, for Afib. Millions of people may have unknowingly experienced Afib.

Some people can tell when they are in Afib, usually from prior experience, but the vast majority cannot or will not admit to themselves that they have some irregularity in their heartbeat. Everyone experiences irregular heartbeats from time to time and most are self limiting or benign, but some are not.

There are many excuses for people who suspect they might have Afib to not go to a doctor to be tested for Afib. For example, setting up and going to an appointment with a doctor (or visiting an emergency room) can be arduous, time consuming, and nerve-wracking. First you have to make appointment with a doctor; then take off work to go; drive to the doctor's office and find a parking place nowhere near the office; wait in the waiting room; wait in an exam room; move to another room to take the EKG; and wait in the exam room for the doctor to provide the results. Given such difficulty, unaware Afib sufferers may prefer to ignore their symptoms or treat themselves rather than seek out testing.

Further, a negative EKG in a doctor's office may not be definitive. A person who has an EKG with negative results may have intermittent or paroxysmal Afib, and the episode may have abated when tested. In these instances the most common current solution is to wear a Holter monitor for 24 to 48 hours. Holter monitors are difficult to put on and to keep in place, and are extremely unpleasant to try to sleep in. Another common approach is to use an “event monitor”, which is worn for up to a month.

Additionally, many people suffer infrequent episodes of Afib with long periods of normal rhythm between episodes. These people are afraid to discontinue their medications, blood thinners and rhythm drugs, because they have no method to easily, and within 48 hours, determine if they are back in Afib. Consequently, they continue taking medication, which is very expensive and carries with it many side effects, some very serious, especially if use is long term.

### SUMMARY OF THE INVENTION

Various inventive embodiments of a portable device for screening for atrial fibrillation are disclosed herein. Inventive embodiments allow easy, inexpensive and rapid determination of whether the subject is experiencing an episode of Atrial Fibrillation or Flutter. Advantageously, a subject may screen him or herself, and given ease and speed of use of an inventive device, may do so on a regular basis without outside diagnostic intervention or requirement of medical or other skill. Any of the inventive embodiments described below can be held in one hand and easily transported by a user.

Inventive devices can be made small, user friendly and easily carried to provide rapid, unlimited, self-administered testing, with an unambiguous display to show a user whether or not they are experiencing an episode of Atrial Fibrillation or Atrial Flutter.

Inventive devices and methods for testing for life-threatening conditions such as Atrial Fibrillation and Atrial Flutter provide numerous advantages, such as:
reasonable price
ease of use
lacks a requirement for external testing and/or analysis in order to determine atrial fibrillation or atrial flutter
ease of understanding of results, which is particularly (though not exclusively) important for seniors, who are at risk to develop atrial fibrillation or atrial flutter
portable
compact
lightweight
easy to carry and diligently use
can be configured to allow targeting to the special needs and medical history of the user
Configuration as a single-purpose device can help reduce false negatives and positives

### FIGURES

FIG. 1 shows an inventive embodiment utilizing an electrocardiogram sensor arrangement.

FIG. 1A shows an inventive embodiment utilizing an electrocardiogram sensor arrangement.

FIG. 2A shows an inventive embodiment utilizing an acoustic sensor arrangement.

FIG. 2B shows an inventive embodiment utilizing an acoustic sensor arrangement.

FIG. 3 shows an inventive embodiment utilizing a pneumatic sensor arrangement.

FIG. 4 schematically shows process and data flow in an inventive embodiment.

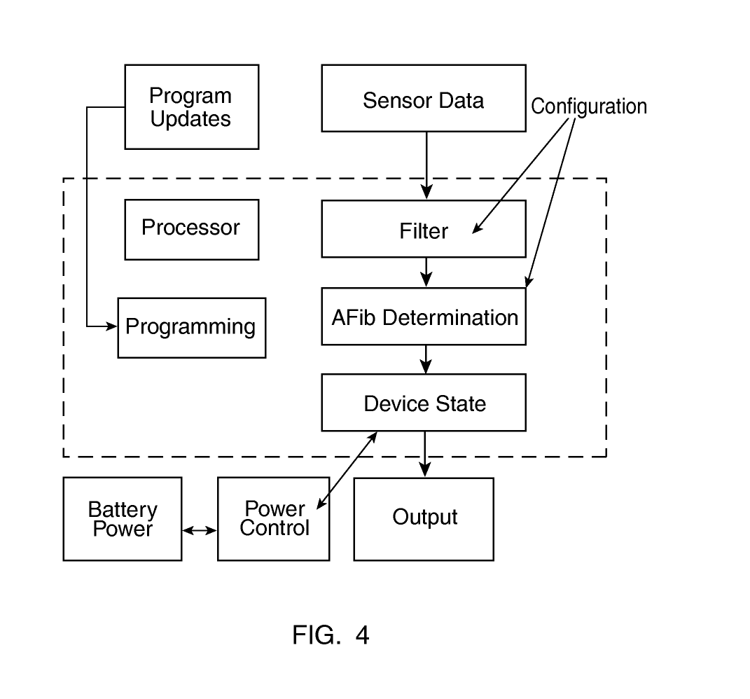

### DETAILED DESCRIPTION OF THE INVENTION

A processor 80 can be programmed to recognize the irregular cardiac patterns that indicate Atrial Fibrillation or Atrial Flutter.

Inventive embodiments allow easy, inexpensive and rapid determination of whether the subject is experiencing an episode of Atrial Fibrillation or Flutter. Advantageously, a subject may screen him or herself, and given ease and speed of use of an inventive device, may do so on a regular basis without outside diagnostic intervention or requirement of medical or other skill. Inventive embodiments described below can be configured to be held in one hand and easily transported by a user.

If you have the experience to recognize the onset of Afib symptoms, you can obtain a prescription for Lavosat and self-administer it until you get to a therapeutic range of blood thinner You can self test for your INR to be sure you are in range.

If sinus rhythm is not restored, you go to your doctor and get a prescription for drugs to return your heart to sinus rhythm. If the drugs are ineffective, you can be cardio-converted or ablated, ablation being the last measure when all else fails.

Throughout this entire Afib episode an inventive device would be helpful.

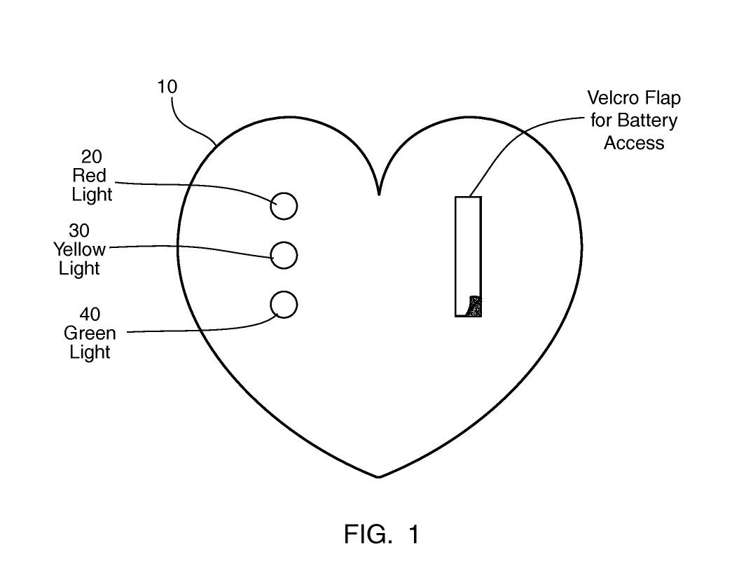

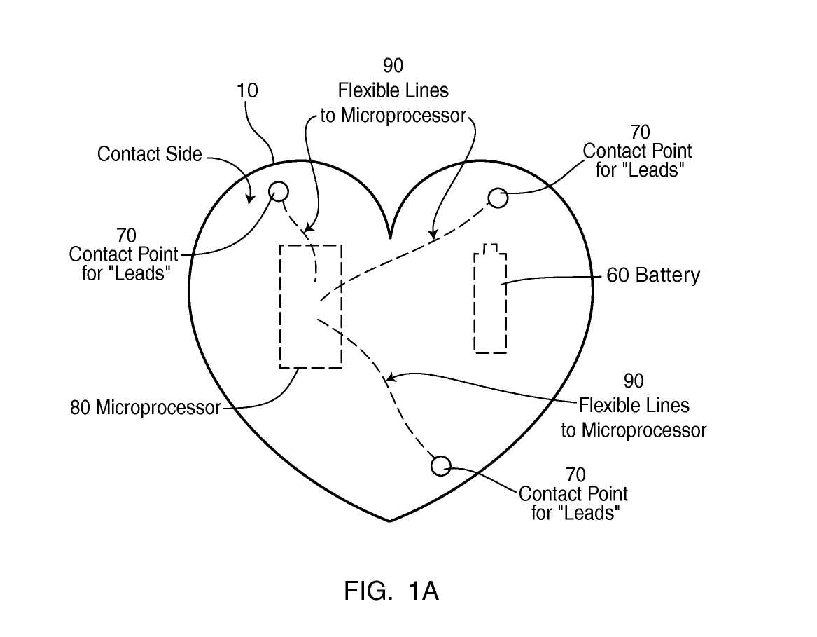

FIG. 1 shows a first inventive embodiment comprising a pad 10, which can be advantageously formed in the shape of a Valentine heart. The pad 10 contains sensors 70 as shown in FIG. 1A, which may be integrated into the skin-facing surface of the pad 10, to detect cardiac electrical impulses. The pad 10 further contains a computing apparatus, such as a processor 80, to determine R-R intervals and the presence or lack of discernible P waves in the QRS complex. The pad 10 also contains at least one indicator output 20, 30, 40 to inform the user whether atrial fibrillation has been detected in the subject. The processor 80 and indicator outputs 20, 30, 40 may also be integrated into the pad_10, advantageously in the body of the pad 10 or on a skin-opposing side.

By using an inventive device, a user is provided, within a brief period, a direct response regarding whether the subject is currently experiencing atrial fibrillation. This response does not require further diagnostic activity or any medical or other skill; if atrial fibrillation is detected, the user is immediately informed of that fact.

In this inventive embodiment the device is an electrocardiographic monitor, and the sensors 70 detecting cardiac electrical impulses comprise three or more leads 90. It has been well settled that three leads 90 are all that are necessary to accurately determine the presence of Atrial Fibrillation or Flutter. An adequately conductive material may be used for the contact points 70 for the leads 90, such as gold, platinum or silver. The leads 90 are attached to the computing apparatus 80, which sends processed data to the indicator outputs 20, 30, 40. No ionizing gel is required, though gel or other means of improving conductive skin contact may be used. An inventive device may be configured so that a result is displayed to the user within 5 to 10 seconds. The device may also be configured to take a longer period of time to gather additional cardiac data.

A subject may receive assistance in using an inventive device from another person, who may apply, activate and read an inventive device and take appropriate action depending on the resulting output. Such assistance may be necessary if the subject is incapacitated.

Other indicator outputs may also be included, such as to inform the user of an error, to replace batteries, or to indicate that atrial fibrillation has not been detected. Indicator outputs may take the form of any easily understood signaling method, such as differently colored lights, differently toned or spaced beeps, different songs, or an LCD or other text or video display.

The pad 10 can also contain a sensor to turn the device on and off when placed on or removed from the skin. Such an automatic on/off sensor can enhance ease of use and prolong battery 60 life. Greater energy efficiency can allow use of smaller batteries 60. Smaller batteries 60, in turn, may make it easier to use a smaller and lighter electronics housing, which can make it easier for elderly or otherwise infirm users to diligently carry and use an inventive device.

An inventive device may be powered by any compatible power source, such as rechargeable batteries 60 or by plugging in to a wall socket.

The Valentine heart shape intuitively provides the user enough information to properly place the device over his or her heart for proper placement of the sensors 70. The ability to place one's hand over one's heart when saying the pledge of allegiance is all the skill required to screen oneself.

Once properly located on the skin, the user is informed within seconds of his or her status. For example, red 20, yellow 30 and green 40 low voltage lights may be used as indicator outputs, with red to indicate the user is experiencing Atrial Fibrillation or Flutter, green to indicate the user is not experiencing Atrial Fibulation or Flutter, steady yellow to indicate improper placement or insufficient data available for processing, blinking yellow to indicate low battery 60 power, and lack of any illumination when active to indicate no battery power or that the device in inoperable. The heart shaped pad and its attachments can comprise the entirety of an inventive device, and can be formed so that it can be rolled up and placed in a tube similar in size and shape to a cigar case.

An inventive device may be configured to allow subject-specific calibration by a physician or otherwise. An inventive device may also have a means for connecting to the Internet or otherwise allowing recorded electrocardiogram data to be transmitted to subject's physician or other monitoring medical personnel.

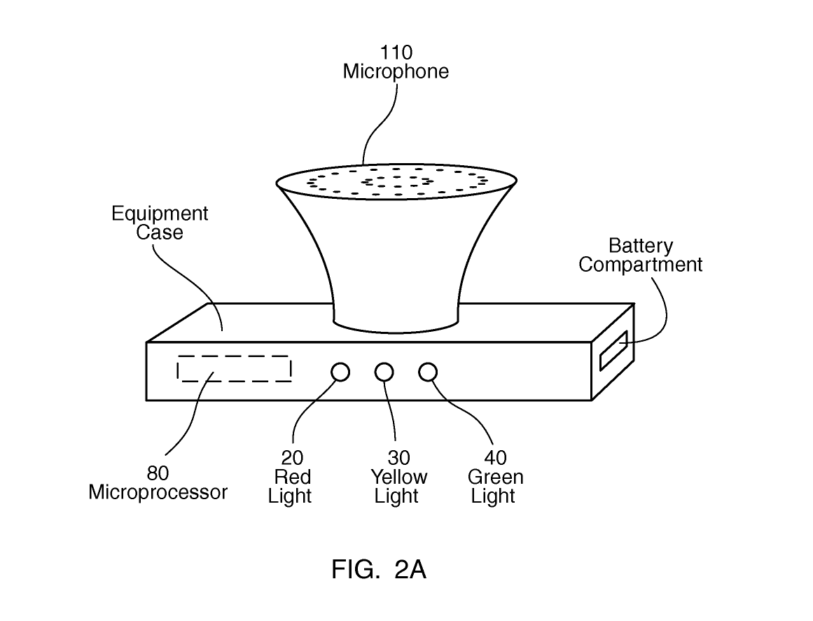

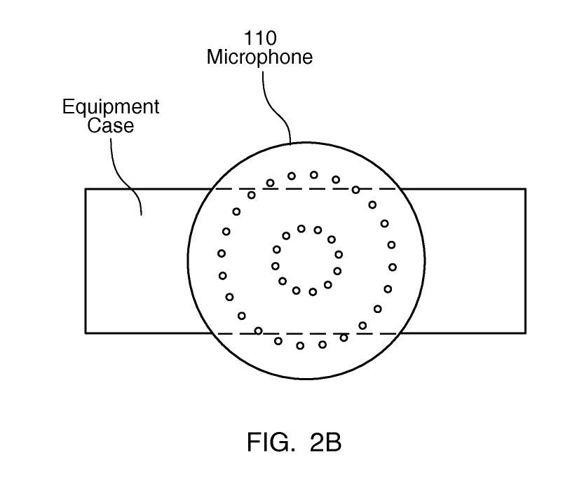

A second inventive embodiment, as shown in FIGS. 2A and 2B, is acoustic and monitors the contractions of the heart via a microphone 110 placed over the heart or on either side of the neck close to the windpipe. Everybody knows these locations and no training is required. It can also be battery operated by readily available batteries.

Once placed an inventive device can be manually or automatically activated. An inventive acoustic device detects the sounds produced by the contractions of the heart, which are sent to a processor for interpretation. The results of interpretation are displayed to the user using simple, unambiguous indicator outputs 20, 30, 40 as described in the first embodiment. Typically, detection of adequate data for reliable indication of atrial fibrillation using acoustics takes 15 to 30 seconds, though more or less time may be taken, depending on, for example, sensor sensitivity, sophistication of analysis software, and a desired level of accuracy.

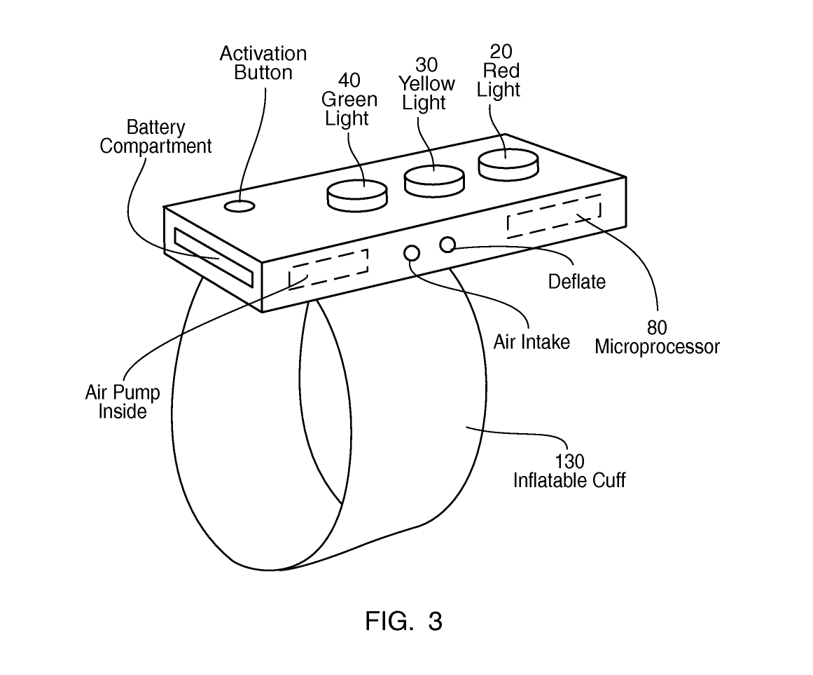

A third inventive embodiment, as shown in FIG. 3, is pneumatic. An inflatable cuff 130 is placed on either of the user's wrists, the device is manually or automatically activated, and the cuff inflates. Cuff inflation of the inventive embodiment is similar to inflation of a blood pressure test cuff; however, an inventive cuff does not slowly deflate as with a blood pressure test. The cuff 130 remains inflated to a pressure that allows the processor to obtain sufficient data regarding heart contractions of the user, via the user's pulse, to determine of whether or not the user is experiencing Atrial Fibrillation or Atrial Flutter.

Generally, sufficient data can be collected using a pneumatic device or method to provide a result to the user within 30 to 60 seconds. The results of interpretation are displayed to the user using simple, unambiguous indicator outputs 20, 30, 40 as described in the first embodiment. Once the result is displayed the cuff can be deflated and removed by the user.

Some inventive embodiments do not require the assistance of trained personnel or the help of a lay person.

Inventive devices can be used standing, sitting or in a supine position.

I claim:

A) Any device, method and/or apparatus as described in any part of the accompanying patent application.

B) Any device, method and/or apparatus as exactly described in the accompanying patent application.
